(19) United States
(12) Patent Application Publication
(10) Pub. No.: US 2014/0358045 A1
(43) Pub. Date: Dec. 4, 2014

(54) MASSAGE DEVICE

(71) Applicant: Blase J. Toto, East Brunswick, NJ (US)

(72) Inventor: Blase J. Toto, East Brunswick, NJ (US)

(21) Appl. No.: 14/294,713

(22) Filed: Jun. 3, 2014

Related U.S. Application Data
(60) Provisional application No. 61/830,291, filed on Jun. 3, 2013.

(51) Int. Cl. A61F 7/00; A61H 99/00

(52) U.S. Cl. CPC A61F 7/00; A61H 99/00
USPC 601/18; 601/137

(57) ABSTRACT

A hand-held massage device is provided for soft tissue treatment, the device operable to be applied with variable amounts of force to the body surface of a patient or subject. The device includes a central portion operable to be gripped by a user, with multiple treatment heads extending from the central portion. The treatment heads may be the same or different to perform a different treatment function. The treatment heads may be removably attachable to the central portion, such as by press or friction fit, screw-type attachment, quick release attachment, or the like. The treatment heads may be subjected to heating or cooling prior to attachment to the central body.

### CROSS-REFERENCE TO RELATED APPLICATIONS

This application claims the benefit of U.S. Provisional Patent application No. 61/830,291 filed Jun. 3, 2013, the entirety of which is incorporated herein by reference.

### FIELD OF THE INVENTION

The invention relates to massage devices.

### BACKGROUND

Massage therapy and chiropractic treatment is a physically demanding activity, requiring significant effort, often for sustained periods of time. It would be advantageous to provide a device which would permit practitioners to administer effective therapy with minimal effort. It would be further advantageous to provide a single massage device capable of performing many functions and administering different types of treatment.

### SUMMARY OF THE INVENTION

In accordance with one embodiment, a massage device is provided for soft tissue treatment. The device is a hand-held device operable to be applied to the body surface of a patient or subject with variable amounts of force. In one embodiment the device includes a central portion operable to be gripped by a user, with multiple treatment heads extending from the central portion. The treatment heads may be the same or different. In this embodiment each of the heads is operable to perform a different treatment function.

In another embodiment the treatment heads of the device are removably attachable to the central portion, such as by press or friction fit, screw-type attachment, quick release attachment, or the like.

The treatment heads may be made of any suitable material such as plastic, rubber, metal, etc. In one embodiment the heads are formed of steel such as stainless steel. Steel is amenable to easy cleaning and disinfecting after each use. Moreover, the steel can be heated or cooled to provide an additional therapy component.

In accordance with one embodiment a massage device is provided which includes a central portion and at least two treatment heads extending therefrom. The massage device may include at least three treatment heads extending from the central portion. One or more of the treatment heads may be removably connectable to the central portion. The central portion may include a grip element.

In one embodiment the central portion includes a hub and a housing disposed around the hub. The hub may include at least one extension extending from the hub, wherein the extension is operable to receive a stem of a head. The housing may include a grip element. The extension may include a receptacle for receiving the stem of the treatment head. The receptacle may be a bore formed in axial alignment with a long axis of the extension. The receptacle may be operable to removably secure a stem.

As will be apparent to those skilled in the art, the central portion is sized and dimensioned to be gripped by a user such that the user's fingers can extend around at least a portion of the central portion. In one embodiment the central portion is roughly triangular in shape, with each side of the triangle being in the range of about 2 inches to about 5 inches in length. In one embodiment, the central portion is approximately the size of the palm of an adult human hand. The thickness of the central portion may be any thickness, but typically is about 1 to about 3 inches in thickness at its thickest cross section.

In another embodiment, a massage system is provided which includes a central portion having a plurality of receptacles, each of the receptacles operable to receive a stem of a treatment head, and a plurality of treatment heads operable to be securably attached to the central portion via the receptacles. One or more of the plurality of treatment heads may be a different shape than the other of the plurality of treatment heads. Different-shaped treatment heads may be used to treat different parts of a patient body. One or more of the treatment heads may be removably attachable to the central portion, and one or more of the treatment heads may be made of a material that can withstand heating and/or cooling treatment, such as refrigeration, boiling, oven heating, etc. Materials such as stainless steel, ceramics and other materials with good temperature retention properties may be employed.

In accordance with still a further embodiment, a method is disclosed for providing a device suitable for massage therapy, the method including providing a massage device including a central portion having a plurality of receptacles, each of the receptacles operable to receive a stem of a treatment head, and a plurality of treatment heads operable to be securably attached to the central portion via the receptacles. The method may include heating or cooling at lease one of the treatment heads.

### BRIEF DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a top plan view of a device in accordance with one or more embodiments of the present invention;

FIG. 2 is a top plan view of a device in accordance with one or more embodiments of the present invention;

FIG. 3 is an elevated perspective view of a device in accordance with one or more embodiments of the present invention;

FIG. 3A is an exploded view of the device according to FIG. 3;

FIG. 3B is an elevated perspective view of a portion of a housing of the device according to FIG. 3 in accordance with one or more embodiments of the present invention;

FIG. 3C is an elevated perspective view of a grip element of the device according to FIG. 3 in accordance with one or more embodiments of the present invention;

FIG. 3D is an elevated perspective view of a core element of the device according to FIG. 3 in accordance with one or more embodiments of the present invention;

FIG. 3E is a perspective view of a treatment head and attachment means of the device according to FIG. 3 in accordance with one or more embodiments of the present invention;

FIG. 4 is an elevated perspective view of a device in accordance with one or more embodiments of the present invention;

FIG. 4A is an exploded view of the device according to FIG. 4; and

FIGS. 5-5F are front views of various treatment heads for fixation to a central portion of a device in accordance with one or more embodiments of the present invention.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

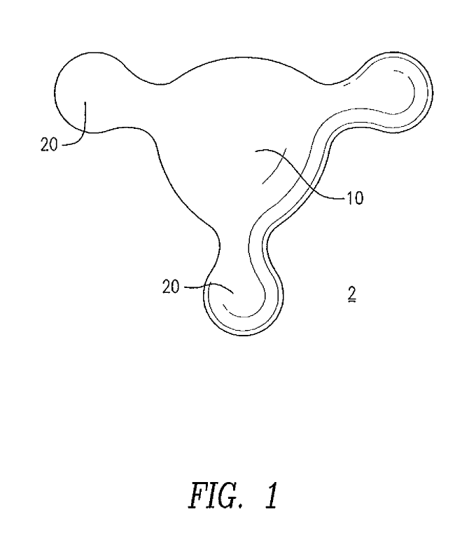

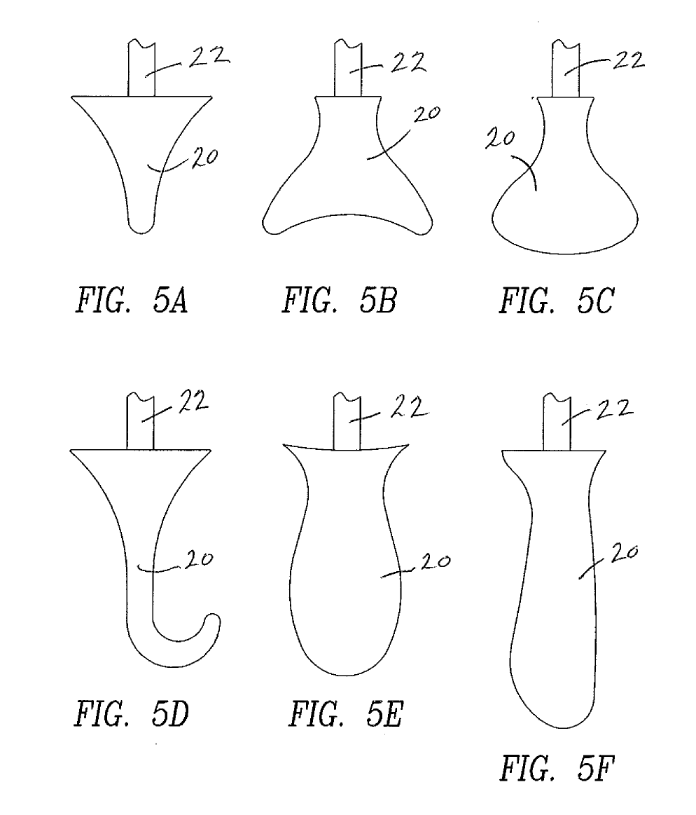

With reference to FIG. 1 an embodiment of a massage device 2 is disclosed, the device 2 including a central portion 10 and heads 20 extending therefrom. In one embodiment the device 2 has a unibody construction. The device may be formed of any suitable material such as rubber, plastic, wood, metal or the like. Each of the heads 20 may be the same, as shown, or different, for example, according to the examples in FIGS. 5-5F. Different heads can perform different functions, as discussed further hereinbelow.

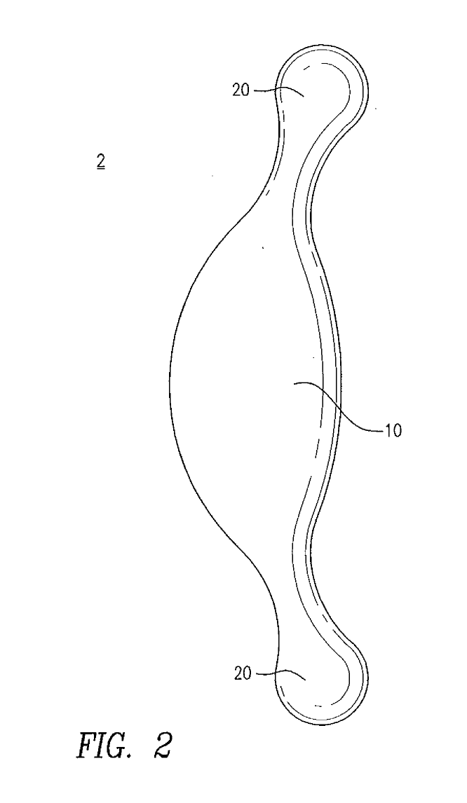

Now referring to FIG. 2, an embodiment of device 2 may include two heads 20 extending from central portion 10. Those skilled in the art will recognize the number of heads extending from central body 10 may be varied.

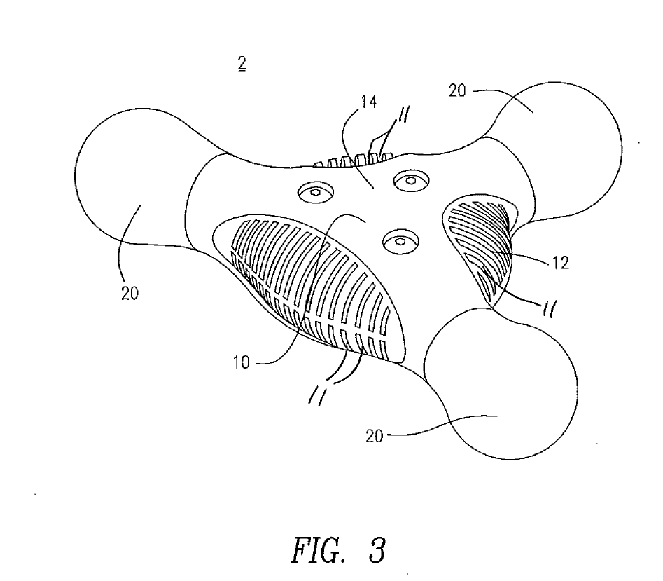

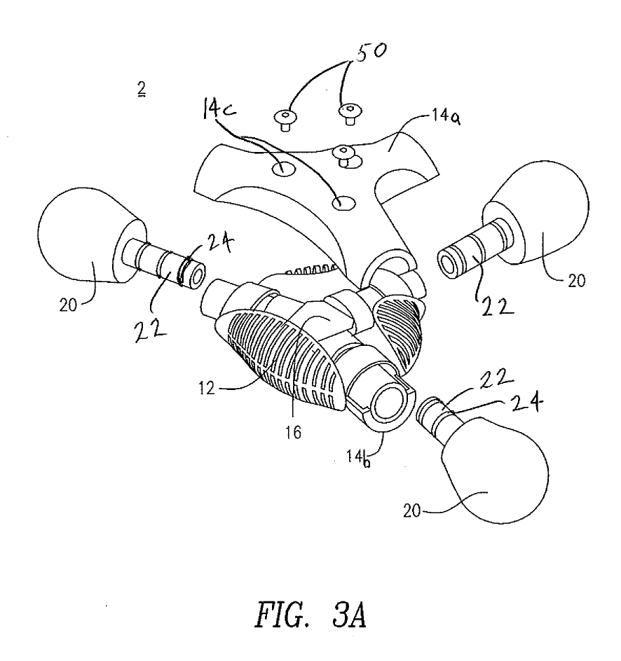

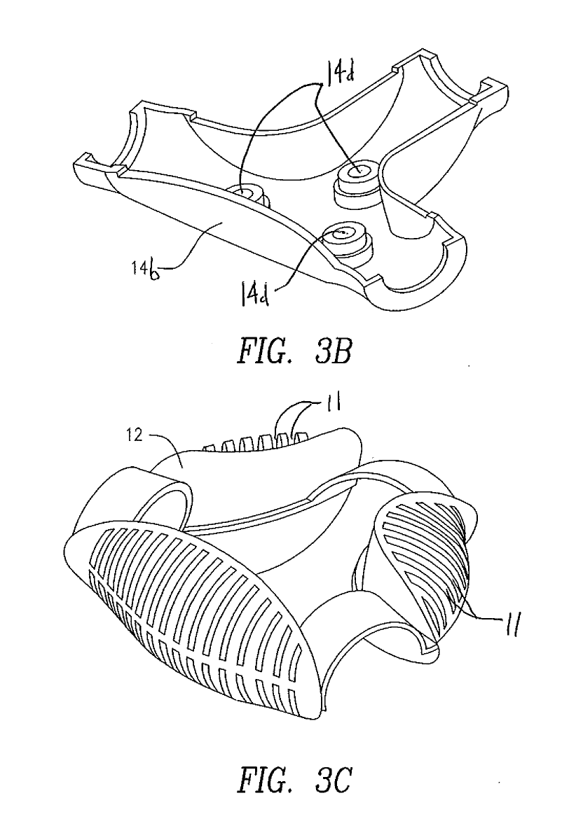

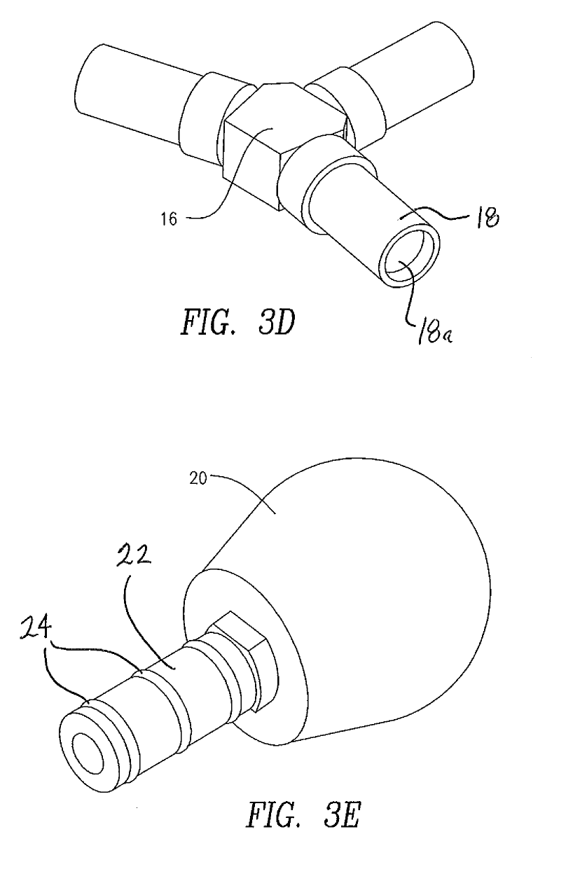

Now referring to FIGS. 3, a device 2 may include central portion 10 with heads 20 extending therefrom. With further reference to FIGS. 3A-3F, central portion 10 may include a grip element 12, housing 14 and hub 16.

Grip element 12 may be composed of any material that provides a comfortable yet secure grip to a user. Suitable materials include elastomeric material, plastic, foam and the like. Grip element 12 may be a soft or resilient element to provide a comfortable, secure grip for a practitioner. The grip element 12 is operable to enable a practitioner to apply firm pressure to a patient without losing a secure grip on the device 2. Moreover, the grip element 2 reduces the likelihood of practitioner fatigue and adverse effects, such as nerve impingement, resulting from pressure of the device 2 on the hand of the practitioner. As shown, the grip element may include vents 11 to facilitate air flow and reduce perspiration which could cause slickness in the grip element 12.

Hub 16 may include extensions 18 having receptacles 18a for removably and securably receiving heads 20. Heads 20 may be formed of rubber, plastic, wood, metal or the like. In one embodiment, a heads 20 includes stem 22 may be removably attachable to the hub 16, by press or friction fit, screw-type attachment, quick release attachment, or the like. In the embodiment shown in FIGS. 3D and 3E, a press fit arrangement is illustrated employing one or more O-rings 24 disposed on a stem 22 of head 20. The hub 16 may include extensions 18 of any suitable length for accommodating heads 20. Heads 20 may be the same or different. In one embodiment the heads 20 are formed of steel such as stainless steel. Steel is amenable to easy cleaning and disinfecting after each use. Moreover, the steel can be heated or cooled to provide an additional therapy component. The heads 20 may be temperature treated separately from the central portion and attached subsequent to temperature treatment. Different heads may be stored in temperature conditions so that they are available for quick connection to the central portion. For example, some heads may be stored in a refrigerator or freezer, while others may be stored in a warmer, and others at room temperature. Thus, the device 2 may be employed with one head being cold, another warm, and another room temperature, all at the same time.

Housing 14 is operable and dimensioned to cover hub 16 to provide a smooth, ergonomic shape for the hand of the practitioner. In one embodiment, the housing 16 is dimensioned to secure the grip element 12 in the device 2. Housing 14 may include a first cover member 14a and a second cover member 14b configured to enclose hub 16 and optionally extensions 18. The second cover member 14b is configured to mate with the first cover member 14a to define a region that accommodates the hub 16. The first cover member 14a and second cover member 14b may be configured to be securely connected to each other, such as by snap-fit, fasteners, etc. For example, first cover member 14a may include apertures 14c and second cover member may include corresponding apertures 14d, wherein the apertures 14c and 14d are alignable and operable to receive a fastener 50 such as a screw or the like. Those skilled in the art will recognize that different types of fasteners may be used. If a removable connection is desired, screws may be employed, and if a nonremovable connection is desired, rivets or the like may be employed.

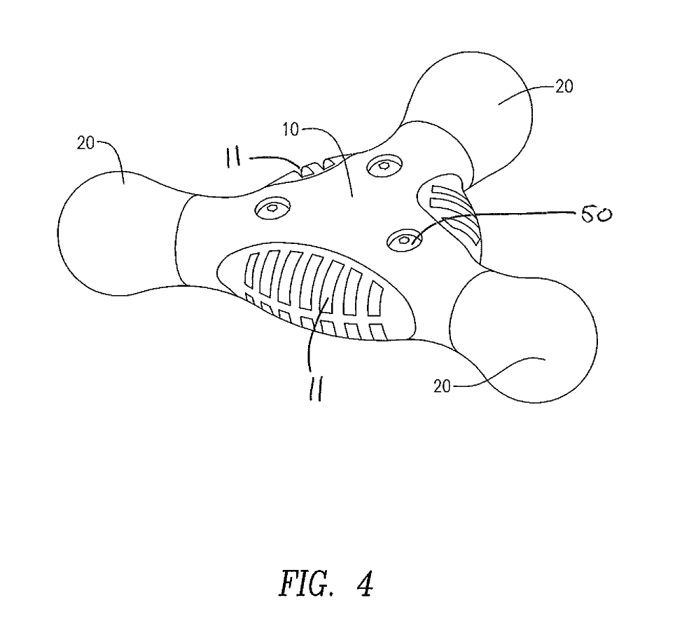

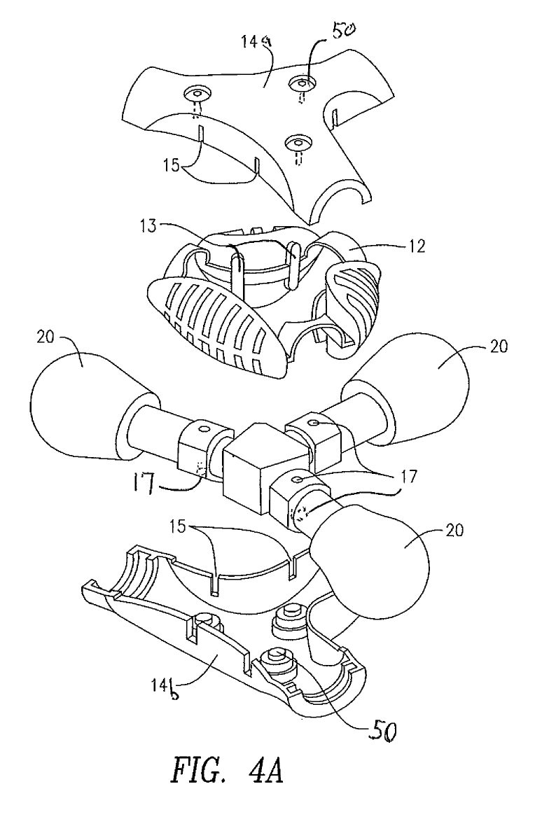

Now referring to FIGS. 4 and 4A, hub 16 may include mounting reinforcement features such as apertures 17 dimensioned to receive fasteners 50. Grip element 12 may include tabs 13 operable to engage capture elements 15 such as slots formed in housing elements 14a, 14b.

Now referring to FIGS. 5A-5F, several embodiments of treatment heads 20 are disclosed which are specialized for particular types of massage therapy and chiropractic treatment. For example, FIG. 5A depicts a configuration usable for working out small muscle trigger points. FIG. 5B depicts a configuration usable for work on convex body parts. FIG. 5C depicts a configuration usable for working on concave body parts. FIG. 5D depicts a configuration usable for working between body parts by pulling. FIG. 5E depicts a configuration which works on all muscles by striping back and forth. FIG. 5F depicts a configuration which works on long muscles.

The foregoing heads are merely exemplary. Those skilled in the art will recognize other treatment heads may be employed with in connection with the presently disclosed subject matter.

Although the devices and systems of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

What is claimed is:

1. A massage device comprising a central portion and at least two heads extending therefrom.

2. The massage device according to claim 1 comprising at least three heads extending from the central portion.

3. The massage device according to claim 1 wherein at least one of the heads is removably connectable to the central portion.

4. The massage device according to claim 1 comprising three heads removably connectable to the central portion.

5. The massage device according to claim 1 wherein at least one of the heads comprises metal.

6. The massage device according to claim 1 wherein at least one of the two heads is different than an other of the at least two heads.

7. The massage device according to claim 1 wherein the central portion comprises a grip element.

8. The massage device according to claim 1 wherein the central portion comprises a hub, and a housing disposed around the hub.

9. The massage device according to claim 8 wherein the hub comprises at least one extension extending from the hub, the extension operable to receive a stem of a head.

10. The massage device according to claim 9, wherein the housing comprises a grip element.

11. The massage device according to claim 9, wherein the extension comprises a receptacle for receiving the stem of the head.

12. The massage device according to claim 11 wherein the receptacle comprises a bore formed in axial alignment with a long axis of the extension.

13. The massage device according to claim 11 wherein the receptacle is operable to removably secure a stem.

14. The massage device according to claim 1 wherein the central portion is sized and dimensioned to be gripped by a user.

15. A massage system comprising a central portion having a plurality of receptacles, each of the receptacles operable to receive a stem of a treatment head, and a plurality of treatment heads operable to be securably attached to the central portion via the receptacles.

16. The system according to claim 15 wherein at least one of the plurality of treatment heads is a different shape than the other of the plurality of treatment heads.

17. The system according to claim 15 wherein at least one of the plurality of treatment heads is removably attachable to the central portion.

18. The system according to claim 15 wherein at least one of the treatment heads is operable to be heated or cooled.

19. A method of providing a device suitable for massage therapy comprising providing a massage device comprising a central portion having a plurality of receptacles, each of the receptacles operable to receive a stem of a treatment head, and a plurality of treatment heads operable to be securably attached to the central portion via the receptacles.

20. The method according to claim 18, further comprising heating or cooling at lease one of the treatment heads.
